(19) United States
(12) Patent Application Publication
(10) Pub. No.: US 2018/0055499 A1
(43) Pub. Date: Mar. 1, 2018

(54) System and Method for Hematoma Formation Prevention

(71) Applicant: TRUMINIM, LLC, Newport Beach, CA (US)

(72) Inventors: Gabor RACZ, Lubbock, TX (US); Ash TAHA, Irvine, CA (US); Vikram PATEL, Algonquin, IL (US)

(73) Assignee: TRUMINIM, LLC, Newport Beach, CA (US)

(21) Appl. No.: 15/690,084

(22) Filed: Aug. 29, 2017

Related U.S. Application Data
(60) Provisional application No. 62/380,952, filed on Aug. 29, 2016.

(51) Int. Cl. A61B 17/00; A61B 17/34; A61M 25/00; A61M 3/02; A61B 17/3203

(52) U.S. Cl. CPC A61B 17/00234; A61B 17/3401; A61M 25/008; A61M 3/0279; A61B 2090/376; A61M 2025/0007; A61B 17/3415; A61B 2017/00778; A61B 17/3203

(57) ABSTRACT

A system and method of performing a procedure for decompression of a spine that includes arranging instrumentalities for performing percutaneous lateral and caudal neural adhesiolysis before or during percutaneous decompression of the spine; and converting high pressure veins to low pressure veins thereby preventing hematoma formation.

### CROSS REFERENCE TO RELATED APPLICATION

This present application claims priority to U.S. Provisional Patent Application No. 62/380,952, filed Aug. 29, 2016, wherein the entire application is incorporated by reference herein in its entirety.

### BACKGROUND

Hematoma formation during a closed percutaneous spinal procedure (e.g. a non-open procedure or surgery), such as in minimally invasive percutaneous lumber decompression of the spinal canal, is a dreaded complication. Spinal stenosis, both central canal and foraminal, are very common conditions of the spine affecting a significant number of people involving all age groups. Many patients are annually diagnosed with varying degrees of spinal stenosis and the number is expected to double by the year 2020.

Spinal stenosis is a disease of the spinal column that is caused by a progressive narrowing of the spinal canal and/or neuroforaminal space thus limiting and restricting the space or room for neural elements. Canal stenosis can be due to the hypertrophy of both posterior and/or anterior elements within the spinal canal. Canal stenosis can also occur due to overgrowth of bone tissue, ligamentum flavum, soft tissue or tumor inside the spinal canal. Additionally, epidural adhesions and fibrosis can occur secondary to several potential etiologies including trauma, surgical trauma, annular tear, infection, hematoma, or intrathecal contrast material. Epidural adhesions and fibrosis are part of the scar tissue that entrap exiting nerve root within the foramen. In addition, this particular fibrosis produces high pressure veins and are a consequence of occluded venous runoff though the neuroforamen. It has been documented that high pressure veins are more likely to bleed if accidently pricked by a needles or other spine procedures.

As a result of spinal stenosis, nerves and/or spinal cord are compressed resulting in pain, tingling, numbness and weakness in the muscles of the affected limb. Additionally, as the stenosis worsens, it has been noted that veins and venous discharge becomes constricted creating high pressure, highly volatile veins. High pressure veins when pricked will bleed because the pressure inside the vein is higher than that of the epidural space and can ultimately lead to the dreaded complication of hematoma formation.

The embodiments of the present disclosure address these and other related needs in the art to prevent hematoma formation.

### SUMMARY

The present solution may be directed towards systems and methods of performing a procedure for decompression of a spine that includes arranging instrumentalities for performing percutaneous lateral and caudal neural adhesiolysis before or during percutaneous decompression of the spine; and converting high pressure veins to low pressure veins thereby preventing hematoma formation. In some embodiments, the step of converting one or more high pressure veins to one or more low pressure veins occurs within a spinal canal. The method may also include percutaneously decompressing one or a plurality of neural elements of the spine including the exiting nerves from the spinal canal and the spinal cord and caudal equina to treat spinal stenosis or other compression pathologies of the spine. In certain embodiments, percutaneously decompressing one or a plurality of neural elements of the spine can occur with or without fluoroscopy.

In some embodiments, a first step of the method can be performing lysis of adhesions before and/or during decompression of the spine. In this respect, the method may include relieving fluid pressure on nerves that are being compressed by the intervertebral foramina; and allowing fluid to pass out a spinal canal thereby avoiding hematoma formation.

In other embodiments, a second step of the method may be percutaneous decompression of the spine.

The method may also be performed in the spinal canal. However, the solution is not so limited and the method may also be performed in the neuroformen at any level of the spine as well as the cervical, thoracic or lumbar spine.

The method may also include advancing a catheter under fluoroscopic guidance through a sacral hiatus and a sacral canal to locate a target fibrosis at a ventral target and/or advancing a catheter under fluoroscopic guidance through a transforaminal approach.

In certain embodiments, the instrumentalities for the method can include an epidural catheter capable of delivering pressurized liquids to the ventral target. The epidural catheter can include a tubular member with a flexible tip. The epidural catheter can be guidable to a target fibrosis at a ventral target. The method in this respect can include depositing, by the epidural catheter, a pressurized liquid solution (e.g. a saline solution) to the ventral target; and allowing fluid to pass out the spinal canal to achieve fluid foraminotomies thereby converting the one or more high pressure veins to one or more low pressure veins. In some embodiments, the step of allowing fluid to pass out the spinal canal can include cleaning, by the liquid solution, one or more adhesions of or proximate the ventral target; providing a space created by cleaning the one or more adhesions; and laterally draining fluids through the space, including the liquid solution, from the ventral target thereby converting the one or more high pressure veins to one or more low pressure veins.

In some embodiments, the step of arranging instrumentalities for performing percutaneous lateral and caudal neural adhesiolysis before or during percutaneous decompression of the spine further includes providing an epidural catheter comprising a tubular member and a flexible tip, the flexible tip being in a first bend position; bending at least a distal end of the flexible tip to a second bend position; and inserting a portion of the epidural catheter, after bending the flexible tip to the second bend position, into the patient into a target fibrosis at a ventral target.

In some embodiments, a method is disclosed for preventing formation of hematomas during performance of a procedure for decompression of a spine. The method can include perforating tissue with an epidural needle; arranging an epidural catheter within a patient through the epidural needle, the epidural catheter being advanced towards a ventral target for performing percutaneous lateral and caudal neural adhesiolysis before or during percutaneous decompression of the spine; cleaning one or more adhesions of or proximate the ventral target, by the epidural catheter, using a pressurized liquid solution, wherein cleaning the one or more adhesions creates a space in place of some or all of the one or more adhesions; and laterally draining fluids through the space, including the liquid solution, from the ventral target thereby converting one or more high pressure veins associated with the ventral target to one or more low pressure veins.

Other aspects and features according to the disclosed technology will become apparent to those of ordinary skill in the art, upon reviewing the following detailed description in conjunction with the accompanying figures.

### BRIEF DESCRIPTION OF THE FIGURES

The present solution will be described by way of exemplary embodiments, but not limitations, illustrated in the accompanying drawings in which like references denote similar elements, and in which:

FIG. 1 depicts a schematic overview of an example implementation under a sacral hiatus approach.

FIG. 2 depicts a schematic overview of an example implementation under a transforaminal approach.

FIG. 3A depicts a view of an example catheter for use in an embodiment of this disclosure.

FIG. 3B depicts a view of the example catheter of FIG. 3A, wherein a distal end of the example catheter has been bent from a first position to a second position.

FIG. 4 depicts a schematic overview of an example implementation of the example catheter of FIGS. 3A-3B under the sacral hiatus approach.

FIG. 5 depicts a table listing exemplary liquid solution volumes for deposition by the epidural catheter.

FIG. 6 depicts an example method of this disclosure.

FIG. 7 depicts an example method of this disclosure.

### DETAILED DESCRIPTION

The following detailed description, reference is made to the accompanying drawings merely showing a possible layout and structure. Any drawings should not be interpreted restrictively. The names of the components are including but not limited to shape, hurt, and response. Unless defined otherwise, all terms of art, notations and other terms or terminology used herein have the same meaning as is commonly understood by one of ordinary skill in the art to which this disclosure belongs. In some cases, terms with commonly understood meanings are defined herein for clarity and/or for ready reference, and the inclusion of such definitions herein should not necessarily be construed to represent a substantial difference over what is generally understood in the art. If a definition set forth in this section is contrary to or otherwise inconsistent with a definition set forth in the patents, applications, published applications and other publications that are herein incorporated by reference, the definition set forth in this section prevails over the definition that is incorporated herein by reference.

Various operations will be described as multiple discrete operations, in turn, in a manner that is most helpful in understanding the present solution. However, the order of description should not be construed as to imply that these operations are necessarily order dependent. In particular, these operations need not be performed in the order of presentation.

In open surgery, the surgeon can visualize any bleeding and cauterize the vessel. In addition, during open surgery, the surgeon can visualize the procedure and decompress the canal and neuroforamen. This is an advantage of open surgery when compared to newer procedures. Current medical practice for treating spinal stenosis has afforded limited viable minimally invasive choices to both practitioners and patients. In mild cases, spinal stenosis can be treated with rest, rehabilitation, strengthening, oral analgesics, anti-inflammatory medications and/or other conservative measures.

Moderate cases of spinal stenosis can be treated temporarily with corticosteroids generally in the form of epidural steroid injections for canal stenosis or transforaminal epidural steroid injections for foraminal stenosis in combination with conservative measures typically with limited or mixed results. Open surgeries are therefore reserved for progressive cases of foraminal stenosis and canal stenosis with variable results. Results depend on the cause of the patient's lower back pain and most patients can expect considerable relief from pain and some improvement in functioning. However, there is some disagreement among surgeons about the success rate of open spine surgeries, which appears to be due to the several factors most notably failed back surgery syndrome (e.g. post-operative scar tissue from open surgery). Minimally invasive surgical procedures and devices have been developed over the years to treat spinal stenosis but with limited success. Typically, such devices have only treated these symptoms by restricting movement and according to some reports with less than 50% of patients reporting some pain relief.

As surgical techniques, procedures and devices have progressed and improved, the trend has moved towards less invasive and minimally invasive procedures and related devices that are preferred by practitioners and patients. There are several companies developing minimally invasive technologies for percutaneous lumbar decompression. For example, procedures practiced by Vertos Medical® of Alta Viejo, Calif., the Vertiflex™ and Totalis@ approaches practiced by VertiFlex, Inc. of San Clemente, Calif., as well as SpineLOOP™ and the EPILOOP™ procedures practiced by SpineLOOP LLC of Costa Mesa, Calif.

As described herein, percutaneous lumbar decompression of the spine (PLDS) is understood as any tool, or tools, used by a practitioner, doctor or surgeon whereby the procedure is minimally invasive and is performed percutaneously to decompress the neural elements to treat the spinal stenosis. PLDS is a closed technique that can be performed under fluoroscopy, MRI, CT, ultrasound, or any mechanism that allows the practitioner, doctor, or surgeon visualization of the anatomy without direct vision to perform the procedure. PLDS can decompress the spine by expanding the canal size or neuroforamen. PLDS can decompress the spine by removing tissue as well.

One drawback for PLDS can be the formation of a hematoma. A hematoma in a closed surgery is a medical emergency as it can compromise the spinal neural elements. For example, the surgeon performing a percutaneous procedure may not have the ability to cauterize the blood vessel or stop the bleeding and this bleeding can lead to a hematoma formation that can cause paralysis and cauda equina syndrome. Additionally, most hematomas and other major complications are associated with the use of sharp needles. See, e.g., Scanlon G. C., et al., Cervical Transforaminal Epidural Steroid Injections More Dangerous Than We Think?, Spine; 32(11):1249-1256. During PLDS, sharp tools such as needles, ronguers and catheters are used to decompress the spine and neural elements. If a high pressure vein is nicked or cut, the vein will bleed and cause a hematoma. Venous run off is most common on the first epidural procedure due to high pressure veins being engorged and large. Following lysis of adhesions (LOA) and fluid foraminotomy, these high pressure veins are converted to low pressure veins and venous run off is less likely. See amison A. E., et. al., Epidural adhesiolysis: an evidence based review, J Neurosurg Sci 2014; 58:65-76. The “Mild® Procedure” by Vertos Medical, Inc. is a type of PLDS was reported to have a case study where indeed the patient developed a hematoma. See Racz G. B. et al., The MILD Procedure, Pain Practice 2013; 13(7):594-596 (hereafter the “Mild® Procedure”). The disclosure of the Mild® Procedure is incorporated herein in its entirety by reference.

Epidural LOA, also known as epidural neuroplasty, fluid foraminotomy and epidural adhesiolysis, is currently used as a minimally invasive technique for the treatment of axial spine or radicular pain when conservative therapy has failed. Although many variations of this procedure exist, most LOA performed today are based on the technique developed at Texas Tech Health Sciences Pain Center and published in 1989. The technique typically involves accessing the epidural space via the sacral hiatus using a large gauge needle and inserting a flexible tipped catheter, such as the epidural catheter described in U.S. Pat. No. 9,539,415 ('415 Patent), a disclosure incorporated herein in its entirety by this reference. The catheter is then advanced to the site of adhesions after an epidurography is performed to map out the adhesions and adhesiolysis via the high-volume administration of saline and medications is performed. The original procedure required that the catheter remain in the epidural space for three days, with the injection of different medications on each of the days.

The technique was subsequently modified to become an ambulatory procedure similar to a traditional epidural steroid injection (ESI), but involving a catheter that is removed immediately following the injection of a combination of a steroid, local anesthetics, and sometimes hyaluronidase and hypertonic saline.

LOA can be performed through the sacral hiatus or at the spinal level through the lumbar transforaminal space using a blunt catheter to floss the neuroforamen. Relative or functional foraminal root entrapment secondary to epidural fibrosis with corresponding nerve root entrapment is frequently evident after an epidurogram and signified by lack of epidural contrast flow into epidural finger projections at those levels. The lysis procedure effectively serves as a fluid foraminotomy reducing foraminal stenosis caused by epidural fibrosis. In addition to increasing foraminal cross-sectional area, adhesiolysis serves to decompress distended epidural venous structures that may exert compression at nearby spinal levels and inevitably cause needle stick related epidural hematomas. Adhesiolysis has led to the development of flexible epiduroscopy that was pioneered by Dr. James Heavner.

The systems and methods disclosed herein resolve these and other problems of the art to avoid hematoma formation during or following PLDS. Specifically, the solution may include a multi-step process for patients who intend to have minimally invasive (e.g. closed) decompression of the spine. The approach may include instrumentalities and steps for performing percutaneous lateral and caudal neural adhesiolysis LOA before or during any form of PLDS. In this respect, high pressure veins can be converted to low pressure veins in the lumbar spine to prevent the potential of hematoma formation. Throughout this disclosure, the term “high pressure” refers to one or more veins that would bleed in the epidural space upon being punctured. In contrast, the term “low pressure” refers to a vein that would not bleed into the epidural space upon being punctured.

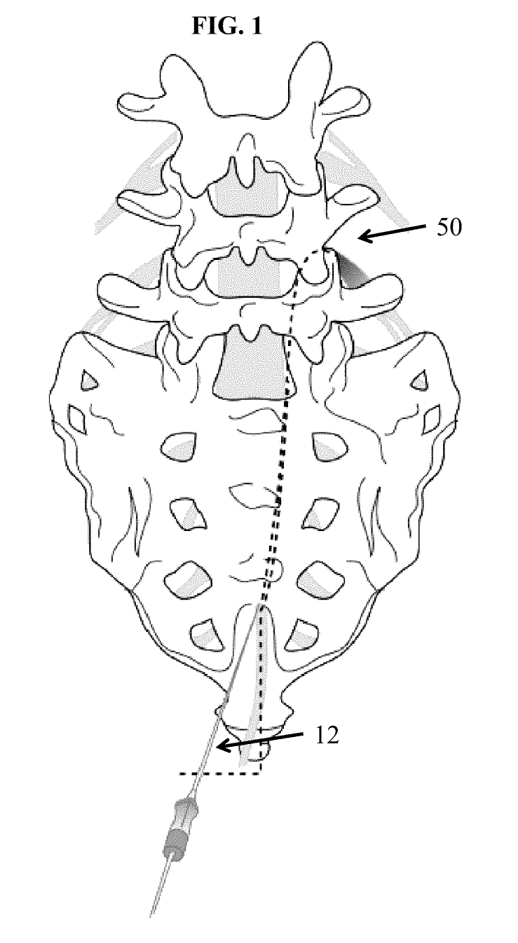

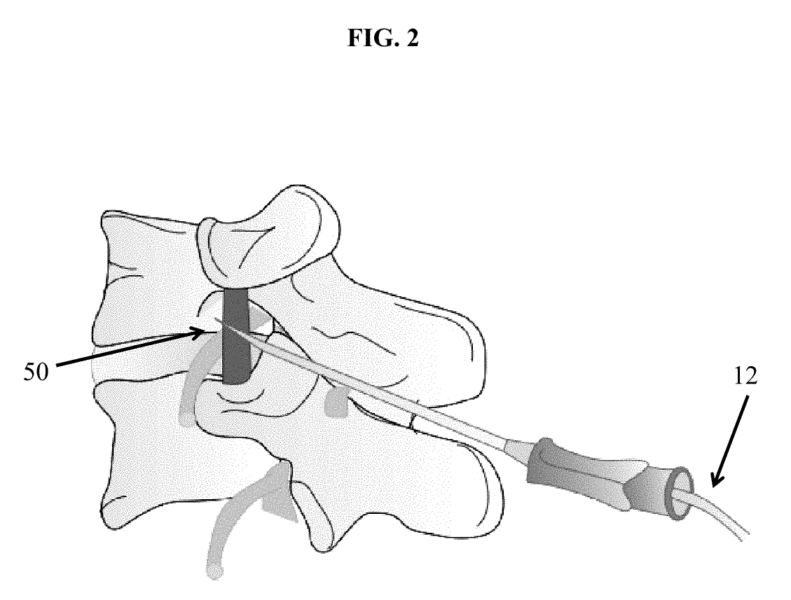

The PLDS that may be used with the herein disclosed systems and methods can include any number of tools, used by a practitioner, doctor and/or surgeon whereby the procedure can be performed percutaneously to decompress the neural elements to treat the spinal stenosis. Certain implementations of the herein disclosed solution includes performing LOA prior to a PLDS approach (e.g. the Mild® process) to achieve fluid foraminotomies and allowing fluid to pass out the spinal canal, venous runoff and ultimately avoiding hematoma formation. The systems and methods disclosed herein can therefore convert high pressure veins to low pressure veins before or during any form of PLDS through a plurality of different approaches including, but not limited to, a sacral hiatus approach of FIG. 1 and a transforaminal approach of FIG. 2.

FIG. 1 specifically depicts an example implementation of certain instrumentalities of this disclosure for converting the one or more veins of high pressure proximate or in communication with a ventral lateral target site of fibrosis and/or adhesiolysis 50 to one or more veins of low pressure. FIG. 1 shows instrumentalities of the solution being arranged, such as arranging of an epidural needle 46, an epidural catheter 12, and/or the like, into a patient to target 50. The direction of the catheter 12 can be just near the midline. Catheter 12 can be driven under guidance (e.g. continuous fluoroscopic guidance) to target 50. Needle 46 rotation, as well as catheter 12 navigation, may need to be used to reach target 50. Needle 46, as described and shown more particularly below, can be initially positioned in the epidural space and then catheter 12 can be advanced towards target 50. The needle 46 can be removed while the catheter 12 can be advanced to target 50, including guidance under fluoroscopy and through sacral hiatus and sacral canal. Once positioned, catheter 12 can be activated so that flow of liquid solutions can be delivered to one or more adhesions. The flow from catheter 12 can be controlled to clean, by a pressurized liquid solution, one or more adhesions that are proximate the target 50.

Turning to FIG. 2, a lateral view is shown of a transforaminal approach once the intertransverse ligament is perforated (e.g. perforated by needle 46). The catheter 12 can be steered to the ventral lateral epidural space. In this embodiment, as the needle 46 is advanced, an indication may be sensed (e.g. a “pop” or other similar vibration) as the needle penetrates the intertransverse ligament. The tip of the needle 46 can be just past the superior articular process in the posterior foramen. In the AP plane, the catheter 12 can be inserted slowly into the foramen and advanced until its tip 26 is just short of the middle of the spinal canal. Once positioned, the catheter 12 can begin cleaning with pressurized liquid solution in order convert the one or more veins from relatively high pressure to relatively low pressure.

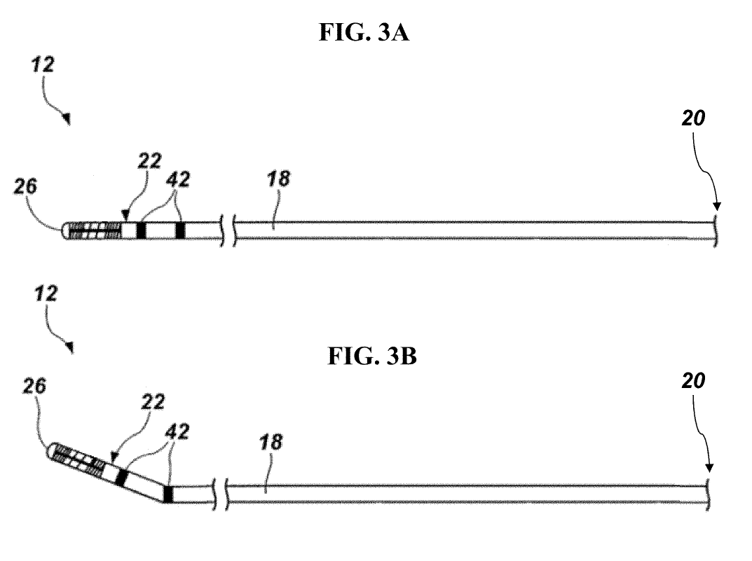

FIG. 3A shows an example epidural catheter 12 for use in the system and method of this disclosure. More specifically, FIG. 3A shows a plan view of catheter 12 having a tubular member 18 extending from a proximal end 20 to a distal end 22. FIG. 3A also shows that catheter 12 can include a flexible tip 26 that can include one or more bend indicators 42 located proximate the distal end 22. These bend indicators can serve as a reference point to bend the tubular member 18 and flexible tip 26 prior to or as catheter 12 is advanced towards target 50. For example, after being provided with catheter 12, the direction in which the catheter 12 can be advanced in the patient and towards the ventral target can be selectively controlled during insertion and placement of the catheter 12 and as the tip 26 is bent. To that end, FIG. 3B shows a plan view of the catheter 12 of FIG. 3B after it has been bent from the first bend position of FIG. 3A to a second bend position of FIG. 3B.

As indicated, the bend indicators 42 can serve as reference points to enable accurate positioning of the catheter 12 and bending of tip 26. For example, catheter 12 can be bent at a location within a predetermine distance from one of the bend indicators 42 (e.g. 5 mm or any other dimension). Further, the catheter 12 can be bent at the location when placing the distal end 22 of the catheter 12 in the epidural space and being advanced towards the target 50.

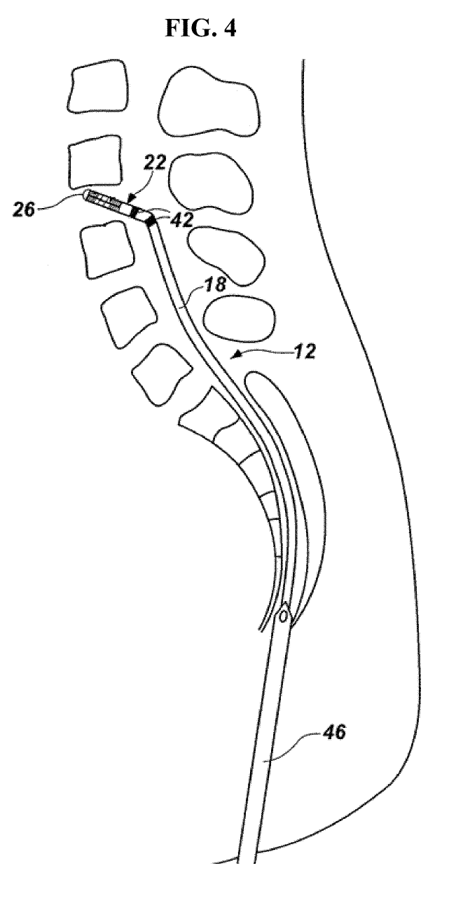

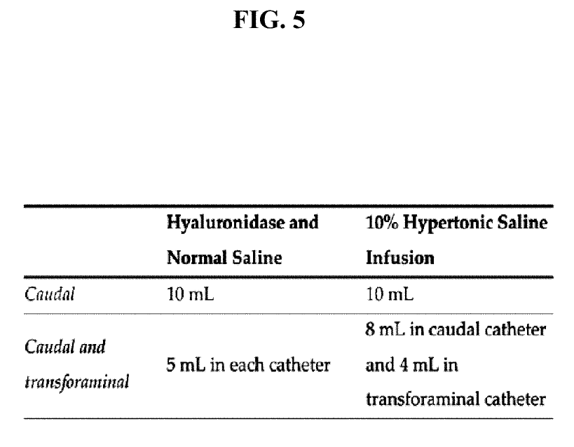

Turning to FIG. 4, a view of the example catheter 12 of FIG. 3 is shown during insertion into the patient and towards the ventral target. After inserting and bending the catheter 12, the procedure of this disclosure as to prevention of formation of hematomas can be performed. For example, the catheter 12 can be inserted into the patient by entering the epidural space (e.g. by puncturing the skin of the patient using an epidural needle 46 that has a perforated, non-blunt tip for perforating tissue). Needle 46 can be moved to a first depth and then the catheter 12, now bent to the second bend position, can be inserted into the patient through the needle 46 and moved towards the target 50. The direction in which the tip 26 of the catheter 12 advances can be controlled by rotation of the catheter 12 at end 20 that is exterior of the patient. If rotated, the bend proximate the distal end 22 can cause the catheter 12 to be capable of continuing to advance towards the ventral target 50. Catheter 12 can then be activated and controlled to clean, by a pressurized liquid solution, one or more adhesions that are proximate the target 50. Fluid type, amount, and type of flow being deposited by the catheter 12 can be manually controlled by the practitioner as needed or required. By cleaning the one or more adhesions, a space can be effectively created where some or all of the one or more adhesions previously existed. The practitioner can use this space to allow for fluids, including the pressured liquid solution, to pass out the spinal canal to achieve fluid foraminotomies. The table of FIG. 5 shows example volume levels of fluids for use in the liquid, pressurized solution for use in converting the one or more high pressure veins to one or more low pressure veins. In so doing, veins previously considered “high pressure” are effectively converted into “low pressure”. Through this novel conversion, formation of hematomas are effectively avoided during the PLDS

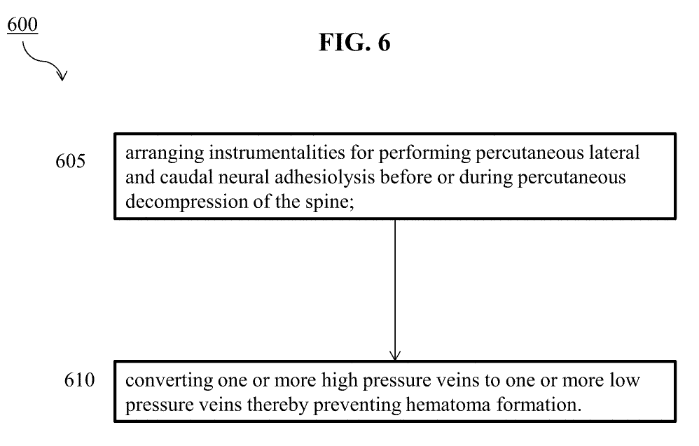

In FIG. 6, a schematic overview of method 600 is disclosed. Step 605 includes arranging instrumentalities for performing percutaneous lateral and caudal neural adhesiolysis before or during percutaneous decompression of the spine. Step 610 includes converting one or more high pressure veins to one or more low pressure veins thereby preventing hematoma formation.

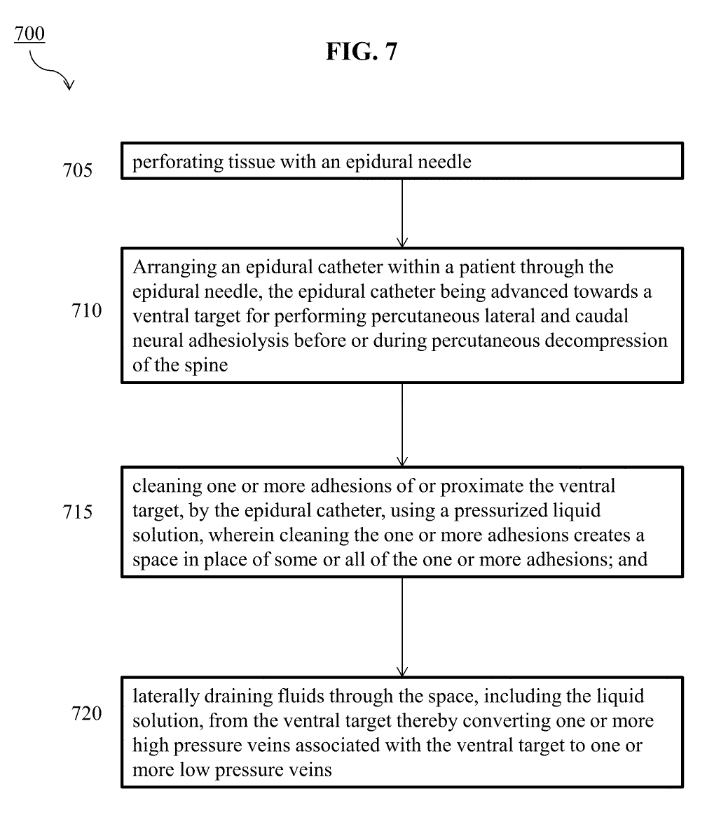

In FIG. 7, a schematic overview of method 700 is disclosed. Step 705 includes perforating tissue with an epidural needle. Step 710 includes arranging an epidural catheter within a patient through the epidural needle, the epidural catheter being advanced towards a ventral target for performing percutaneous lateral and caudal neural adhesiolysis before or during percutaneous decompression of the spine. Step 715 includes cleaning one or more adhesions of or proximate the ventral target, by the epidural catheter, using a pressurized liquid solution, wherein cleaning the one or more adhesions creates a space in place of some or all of the one or more adhesions. Step 720 includes laterally draining fluids through the space, including the liquid solution, from the ventral target thereby converting one or more high pressure veins associated with the ventral target to one or more low pressure veins.

In one implementation the practitioner may decompress the spine and flood a predetermined area with a pressurized liquid solution so as to clean a side of vertebral body. Once adhesions have been cleaned, then there may be space whereby fluids may drain laterally thereby reducing the pressure of the corresponding vein. In turn, hematoma formation may be prevented by this conversion from high to low pressure.

Other features and advantages of the solution will be apparent from the description herein. The examples are provided herein are solely to illustrate the solution by reference to specific embodiments. These exemplifications, while illustrating certain specific aspects of the solution, do not portray the limitations or circumscribe the scope of the disclosed system and methods. Many variations to those described above are possible

1. A method of performing a procedure for decompression of a spine, the method comprising:
arranging instrumentalities for performing percutaneous lateral and caudal neural adhesiolysis before or during percutaneous decompression of the spine; and
converting one or more high pressure veins to one or more low pressure veins thereby preventing hematoma formation.

2. The method of claim 1, wherein converting one or more high pressure veins to one or more low pressure veins occurs within a spinal canal.

3. The method of claim 1, further comprising: percutaneously decompressing one or a plurality of neural elements of the spine including the exiting nerves from the spinal canal and the spinal cord and caudal equina to treat spinal stenosis.

4. The method of claim 3, wherein percutaneously decompressing a plurality of neural elements is performed with fluoroscopy.

5. The method of claim 3, wherein percutaneously decompressing a plurality of neural elements is performed without fluoroscopy.

6. The method of claim 1, wherein the instrumentalities comprise an epidural catheter comprising a tubular member with a flexible tip, the epidural catheter being guidable to a target fibrosis at a ventral target, the method further comprising:
depositing, by the epidural catheter, a pressurized liquid solution to the ventral target; and
allowing fluid to pass out the spinal canal to achieve fluid foraminotomies thereby converting the one or more high pressure veins to one or more low pressure veins.

7. The method of claim 6, wherein the allowing fluid to pass out the spinal canal comprises:
cleaning, by the liquid solution, one or more adhesions of or proximate the ventral target;
providing a space created by cleaning the one or more adhesions; and
laterally draining fluids through the space, including the liquid solution, from the ventral target thereby converting the one or more high pressure veins to one or more low pressure veins.

8. The method of claim 1, wherein the arranging instrumentalities for performing percutaneous lateral and caudal neural adhesiolysis before or during percutaneous decompression of the spine further comprises:
providing an epidural catheter, the epidural catheter comprising a tubular member and a flexible tip, the flexible tip being in a first bend position;
bending at least a distal end of the flexible tip to a second bend position; and
inserting a portion of the epidural catheter, after bending the flexible tip to the second bend position, into the patient into a target fibrosis at a ventral target.

9. The method of claim 1, wherein a first step of the method is performing lysis of adhesions before or during decompression of the spine.

10. The method of claim 9, further comprising:
relieving fluid pressure on nerves that are being compressed by the intervertebral foramina; and
allowing fluid to pass out a spinal canal thereby avoiding hematoma formation.

11. The method of claim 9, wherein a second step of the method is percutaneous decompression of the spine.

12. The method of claim 1, wherein the method is performed in the spinal canal.

13. The method of claim 1, wherein the method is performed in the neuroformen at any level of the spine.

14. The method of claim 1, wherein the method is performed in the cervical, thoracic or lumbar spine.

15. The method of claim 1, further comprising: guiding an epidural catheter under fluoroscopic guidance through a sacral hiatus and a sacral canal to locate a target fibrosis at a ventral target.

16. The method of claim 1, further comprising: guiding an epidural catheter under fluoroscopic guidance through a transforaminal approach.

17. A method of preventing formation of hematomas during performance of a procedure for decompression of a spine, the method comprising:
perforating tissue with an epidural needle;
arranging an epidural catheter within a patient through the epidural needle, the epidural catheter being advanced towards a ventral target for performing percutaneous lateral and caudal neural adhesiolysis before or during percutaneous decompression of the spine;
cleaning one or more adhesions of or proximate the ventral target, by the epidural catheter, using a pressurized liquid solution, wherein cleaning the one or more adhesions creates a space in place of some or all of the one or more adhesions; and
laterally draining fluids through the space, including the liquid solution, from the ventral target thereby converting one or more high pressure veins associated with the ventral target to one or more low pressure veins.

18. A method of preventing formation of hematomas during performance of a procedure for decompression of a spine, the method comprising:
perforating tissue with an epidural needle;
arranging an epidural catheter within a patient through the epidural needle, the epidural catheter being advanced towards a ventral target for performing percutaneous lateral and caudal neural adhesiolysis before or during percutaneous decompression of the spine;
cleaning one or more adhesions of or proximate the ventral target, by the epidural catheter, using a pressurized liquid solution, wherein cleaning the one or more adhesions creates a space in place of some or all of the one or more adhesions; and
laterally draining fluids through the space, including the liquid solution, from the ventral target thereby converting one or more high pressure veins associated with the ventral target to one or more low pressure veins.
